(19) United States
(12) Patent Application Publication
(10) Pub. No.: US 2026/0139209 A1
(43) Pub. Date: May 21, 2026

(54) SYSTEM, APPARATUS AND METHOD FOR A MODULAR PHOTOBIOREACTOR

(71) Applicant: SOLARFERM LIMITED, Auckland (NZ)

(72) Inventors: Gabriel O’Sullivan JAMES, Auckland (NZ); Tancred Giles FRICKEY, Rotorua (NZ)

(73) Assignee: SOLARFERM LIMITED, Auckland (NZ)

(21) Appl. No.: 19/474,892

(22) PCT Filed: Apr. 15, 2024
(86) PCT No.: PCT/IB2024/053686

§ 371 Date: Oct. 13, 2025

Related U.S. Application Data
(60) Provisional application No. 63/459,471, filed on Apr. 14, 2023.

(51) Int. Cl. C12M 1/00

(52) U.S. Cl. CPC C12M 21/02; C12M 23/02; C12M 23/22; C12M 31/10; C12M 41/10

(57) ABSTRACT

An illumination system for use in a photobioreactor containing photosynthetic microorganisms, has an array of poly gonal prism cells, each of said polygonal prism cells comprising a plurality of substantially flat rectangular panels, each panel having a top edge, a bottom edge, and lateral edges and a flat surface therebetween, wherein said panels are configured to provide illumination at said flat surface, and further configured to be connected to at least one other of said panels along each said lateral edge, and wherein the top and bottom of the cells formed by the respective top and bottom edges of the panels thereof are substantially open, thereby permitting flow therethrough of a fluid containing said photosynthetic microorganisms to be illuminated at the flat surfaces of said panels.

### FIELD OF INVENTION

The present invention relates to a photobioreactor for growing algae.

### BACKGROUND OF INVENTION

Concerns about climate change, carbon dioxide (CO₂) emissions, food security and depleting mineral oil and gas resources have led to widespread interest in the production of biofuels, biomass and nutrients from cultivated photosynthetic microorganisms, such as microalgae and cyanobacteria. As compared to other plant-based feedstocks, microalgae and cyanobacteria have higher CO₂ fixation efficiencies and growth rates, and growing photosynthetic microorganisms can efficiently utilize wastewater and industrial gases as nutrient sources. The most developed method for extracting biofuels from microalgae is converting their stored lipids into renewable fuel.

Microalgae and cyanobacteria are classified as photoautotrophic organisms, or organisms that can survive, grow and reproduce with energy derived entirely from the sun through the process of photosynthesis, which is essentially a carbon recycling process through which inorganic CO₂ is combined with solar energy, other nutrients, and cellular biochemical processes to synthesize carbohydrates and other compounds that are critical to life. Photoautotrophic culture is a technically and economically feasible way to produce photosynthetic microorganism biomass at commercial scale.

Algae biomass is generally grown in a water slurry contained in a photobioreactor, which is typically a closed container, using photosynthetic microorganisms. Effective photobioreactor designs must be able to sustain high-density cultures while enabling efficient light delivery and gas exchange. Most types of photobioreactors used in algal cultivation face fundamental limitations in delivery of sufficient light to maintain high photosynthetic rates, such that light availability proves to be the most limiting factor for photosynthetic microorganism growth.

One objective of photosynthetic microorganism culture is, thus, to provide sufficient and uniform quantity of light to each photosynthetic microorganism; however, light is quickly absorbed by the surface-exposed photosynthetic microorganisms. The higher the concentration of photosynthetic microorganisms, the more light is absorbed. As a result, photosynthetic microorganism cells close to the light source receive far more light than photosynthetic microorganisms in darker regions, leaving those photosynthetic microorganisms in the darker regions with insufficient light for growth. Uneven light distribution causes the algae to be overexposed at the surface and underexposed below the light penetration depth.

There is a need for new highly scalable and efficient bioreactors capable of generating large quantities of biomass to meet current food, energy and environmental challenges.

In addition, there exists a need for photobioreactors that facilitate ease of installation and illumination of photosynthetic microorganisms with sufficient and uniform light.

### SUMMARY OF INVENTION

The modular photobioreactor according to embodiments of the invention solves a fundamental limitation of photosynthesis the attenuation of light. With the modular use of illumination materials, the light path length has been optimized for the delivery of light into the liquid growth medium. The photobioreactor is comprised of modular units, e.g., flat panels, that can be constructed to form polygonal prism shapes for being inserted to deliver either or both natural light and/or artificial light into the liquid growth medium.

The embodiments of the invention may utilize lightweight flexible luminous thin film assembled into open-ended polygonal tubular cell structures that provide internal illumination within the bioreactor, which may be interconnected horizontally and/or vertically to form larger structures.

According to embodiments of the invention shown in the figures, the polygonal tubular cell structure formed by the flat modular panels may be in the shape of a hexagon. However, it will be understood that other embodiments of the invention may utilize different polygonal shapes within the scope of the invention, including for example, triangular, square, rectangular, hexagonal, and/or octagonal prisms (or combinations thereof) that may be repeated by interconnection and repeated regularly to produce the desired size illumination system for a containment vessel in which the reaction takes place.

The open-ended hollow polygonal tubular cell structure according to embodiments of the invention may allow liquid and/or gas to flow through the tubular cell structure, thereby providing an optimized environment for light delivery to microalgae and photosynthetic growth.

The geometry and internal diameter of the polygonal cell structure according to embodiments of the invention is optimized for hydrodynamics, photonics and photosynthetic growth.

The open tubular cell structures according to embodiments of the invention may be assembled into high density honeycomb lattice structures which may be configured to be immersed within closed tanks and/or other containment vessels containing the photosynthetic microorganisms.

Among the advantages of the lightweight flexible thin film lattice structures according to embodiments of the invention include their ability to be assembled into open-ended tubular modules that can be scaled horizontally and/or vertically through the addition of more cells or modules as needed. The interconnection between modular panels may include optical connections for light to pass from one panel to another.

Another advantage of the polygonal cell structures according to embodiments of the invention include their versatility to allow the luminous thin film modular cells to be efficiently packed at high density in order to minimize thin film surface area wastage and to generate substantially uniform light emission across the thin film surface area.

According to embodiments of the invention, the luminous thin film modular cells may be passive devices that function as light guides to guide light from one or more external light sources connected through the optical ports of the thin film tubular cells or thin film tubular modules.

According to embodiments of the invention, the external light sources may be artificial light and/or natural light, e.g., sunlight or daylight.

According to embodiments of the invention utilizing external light, the system may include a wavelength filtering module to remove light wavelengths that are not usable for photosynthetic growth and would otherwise result in waste heat generation.

According to embodiments of the invention, utilizing external light, the system may further include a wavelength shifting module to increase the amount of usable light for photosynthetic growth and thereby decrease waste heat generation.

According to some embodiments, the wavelength filter or wavelength shifting modules may be embedded in one or more of the thin films of the modular panels to increase the usable light for photosynthesis and decrease waste heat generation.

According to embodiments of the invention, the luminous thin film panels may be active devices that include light emitting elements receiving power through power ports of the interconnection port of the modular panels of the thin film tubular cells or thin film tubular modules. According to embodiments of the invention, the light source may be one or more of light emitting diodes (LEDs), and/or printable organic light emitting diode (OLEDs), including micro-or nano-LEDs or OLEDs or polymer light-emitting diode (PLEDs, P-OLEDs) or active-matrix organic light emitting diodes (AM-OLEDs) or electro-emissive quantum dot light emitting diodes (QD-LEDs, ELQDs, EL-QLEDs, QDELs) or photo-emissive quantum dot light emitting diodes (QLEDs, QD-OLEDs), which may be deposited directly onto the thin film of the modular panel.

Embodiments of the invention may be hybrid devices comprised of both passive light guides utilizing external light and active light emitting elements.

The luminous thin films devices according to embodiments of the invention may be transparent, thereby enabling light to be emitted through one surface or both surfaces of the cell.

According to embodiments of the invention, emission of light through both surfaces of the thin film structure may enable more efficient manufacturing and fabrication of cells or modules as only a single wall of the thin film is required to assemble the lattice structure.

According to embodiments of the invention, emission of light through both surfaces of the thin film cell walls may enable more efficient light delivery through reducing light absorption losses between the layers within the thin films.

Embodiments of the invention may include mechanisms for avoiding fouling of the panels during use. According to embodiments of the invention, the surfaces of the thin film walls may be coated with transparent compositions and layers of antistatic or antifouling materials to prevent fouling and reduce light attenuation on the surface thin film walls.

According to embodiments of the invention, the surfaces of the thin films may be coated with transparent compositions of materials and structures that create superhydrophobic surfaces that trap gas bubbles to create a gaseous plastron at the solid-liquid interface of the thin film walls to reduce light attenuation from fouling on the surface of the thin film walls.

According to embodiments of the invention, the surfaces of the thin film modular cells may be coated with transparent compositions of gels or beads that increase the surface area for light delivery and reduce light attenuation from biomass in the liquid culture.

According to embodiments of the invention, the surfaces of the thin films may be coated with transparent compositions of gels or beads that increase the porous surface area for gaseous plastron formation to reduce light attenuation from fouling on the surface of the thin film walls.

Embodiments of the invention may include a gas delivery module and/or sparging system to aerate the thin film modular cells using one or more gas compositions to agitate the culture and mix the photosynthetic microorganisms to reduce light attenuation from biomass in the liquid culture.

Embodiments of the invention may include a gas delivery module and/or sparging system to aerate the thin film modular cells using one or more gas compositions to establish and replenish the gaseous plastron at the solid-liquid interface, thereby reducing light attenuation from fouling on the thin film surface.

Embodiments of the invention may include a gas delivery module and/or sparging system to aerate the thin film modular cells using one or more gas compositions and sparging systems to increase light penetration into the liquid culture through the use of gas bubbles.

According to embodiments of the invention, the gas delivery module and/or sparging system may add or mix transparent gels, beads or surfactants into the gas compositions in order to reduce gas flow rates and increase the gas bubble stability and gas bubble residence time, thereby increasing light penetration into the liquid culture.

According to embodiments of the invention, the gas delivery module and/or sparging system may add or mix transparent gels, beads or surfactants to reduce gas flow rates and increase the gas bubble stability and residence time and increase light penetration into the liquid culture.

It will be understood that, while some applications are discussed in detail herein, the thin film modular cells according to embodiments of the invention may be deployed in a wide range of configurations including filtration systems, fermenters and bioreactors for batch and continuous cultivation.

The thin film modular cells according to embodiments of the invention may be deployed into container tanks that provide the structural support for the liquid medium. The use of tanks allows for the use of light weight flexible thin film materials in the cells. The modular cells can be easily interchanged for different functions and operating conditions. The modular arrays of cells can easily added and removed from tanks. The modular cells can massively scale both horizontally and vertically for industrial scale biomass production.

The use of natural light in conjunction with embodiments of the invention may be suited for high efficiency carbon capture and biomass production. The use of artificial light in conjunction with embodiments of the invention may be suited for high productivity and high-density biomass production. The tanks can be deployed across non-arable land, in oceans and water ways, and collocated next to heavy industry carbon emission sites.

The modular photobioreactor according to embodiments of the invention may be used to grow a wide range of photosynthetic microorganisms including, but not limited to the microalgal classes Cyanophyceae (blue-green algae), Chlorophyceae (green algae), Bacillariophyceae (including the diatoms), and Chrysophyceae (including golden algae).

### BRIEF DESCRIPTION OF THE DRAWINGS

The subject matter regarded as the invention may be understood by reference to the following detailed description, when read together with the accompanying drawings, in which:

FIG. 1 depicts a top or bottom view of several possible embodiments of a basic geometry of the photobioreactor cell according to embodiments of the invention.

FIG. 2 depicts a side view of several possible embodiments of a basic geometry of the photobioreactor cell according to embodiments of the invention.

FIG. 3 depicts an isometric view of several possible embodiments of a basic geometry of the photobioreactor cell according to embodiments of the invention.

FIG. 4 depicts a top or bottom view of several possible embodiments of an array configuration of multiple photobioreactor cells according to embodiments of the invention.

FIG. 5 depicts a top or bottom view of several possible embodiments of an array configuration of multiple photobioreactor cells according to embodiments of the invention.

FIG. 6 depicts a top or bottom view of a possible embodiment of a pilar array configuration of multiple photobioreactor cells according to embodiments of the invention.

FIG. 7 depicts a top or bottom view of several possible embodiments of an array module configuration of multiple photobioreactor cells within a protective structure according to embodiments of the invention.

FIG. 8 depicts a isometric view of several possible embodiments of an array module configuration of multiple photobioreactor cells within a protective structure according to embodiments of the invention.

FIG. 9 depicts a wall of a light guide cell according to embodiments of the invention.

FIG. 10 depicts walls of a hexagonal light guide cell according to embodiments of the invention.

FIG. 11 depicts an assembled light guide cell according to embodiments of the invention.

FIG. 12 depicts light transmission and emission of light guide cell according to embodiments of the invention.

FIG. 13 depicts a thin film assembly of a light guide according to embodiments of the invention.

FIG. 14 depicts layers of a thin film assembly light guide according to embodiments of the invention.

FIG. 15 depicts a wall of a LED cell according to embodiments of the invention.

FIG. 16 depicts walls of a hexagonal LED cell according to embodiments of the invention.

FIG. 17 depicts an assembled hexagonal LED cell according to embodiments of the invention.

FIG. 18 depicts an assembled printable light emitting device according to embodiments of the invention.

FIG. 19 depicts an assembled printable light emitting device according to embodiments of the invention.

FIG. 20 depicts light transmission and emission of printable light emitting devices according to embodiments of the invention.

FIG. 21 depicts light emission from thin film printable light emitting devices according to embodiments of the invention.

FIG. 22 depicts a stack of layers of printable light emitting devices in a thin film assembly according to embodiments of the invention.

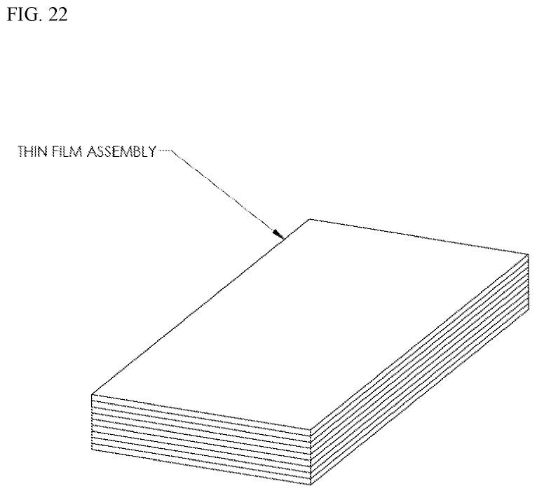

FIG. 23 depicts a stack of layers of printable light emitting devices in a thin film assembly according to embodiments of the invention.

FIG. 24 depicts a stack of layers of printable light emitting devices in a thin film assembly according to embodiments of the invention.

### DETAILED DESCRIPTION OF THE INVENTION

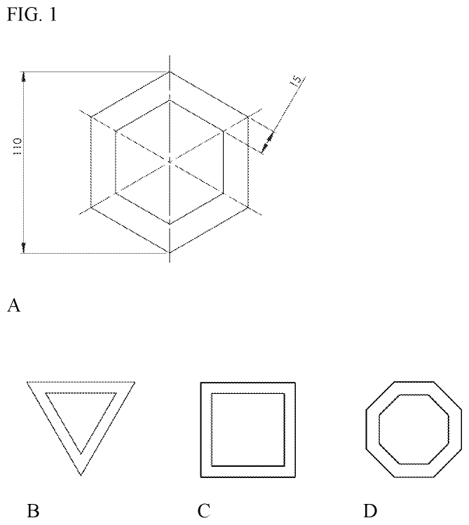

A photobioreactor cell in one embodiment as described herein can have an elongated annular or tubular shape. Among the possible configurations of the photobioreactor cell with an elongated annular or tubular shape is a polyhedron prism, with a cross-section selected from the group consisting of triangle, square, hexagon, and octagon. FIGS. 1-3 show several possible configurations, such a triangular prism, a rectangular prism, a hexagonal prism and an octagonal prism.

FIG. 1 shows a geometry of a photobioreactor cell according to an embodiment of the invention in a schematic top/bottom view of the modular photobioreactor cell showing the basic geometry for optimised growth conditions, and optimised for light transfer to the liquid medium, and optimised for mass transfer of nutrients between liquid and gas phases. The internal diameter of the photobioreactor cell may be sized to meet the light requirements for the optimal photosynthetic growth rate and/or cultivation methods of specific photosynthetic microorganisms. Fast growing cyanobacteria may have photosynthetic growth rates of between 0.5 to 1.0 doublings per hour under optimal conditions. Slow growing microalgae may have photosynthetic growth rates of between 0.5 to 1.0 doublings per day under optimal conditions. The internal diameter of the photobioreactor cell may be, but not limited to, between 50 mm and 250 mm depending upon the light requirements and, or cultivation methods. The surface to volume ratio has been optimised for light transfer into the liquid medium embodiment examples include, but not limited to, 25:1, 50:1, 75:1 and 100:1 m²/m³. The diameter of the cell wall may be sized to meet the physical characteristics of the materials, functional characteristics of passive or active device, and/or structural requirements of the photobioreactor cell. The diameter of the cell wall may be between, but not limited to, 0.1 mm to 15 mm, depending upon thin film device type.

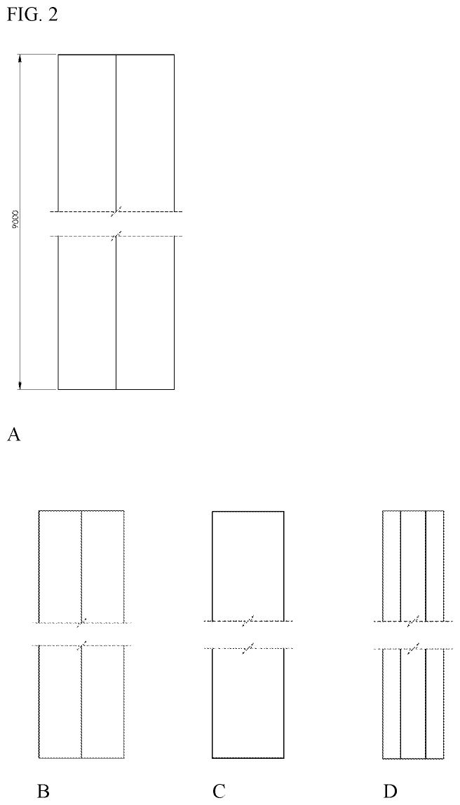

FIG. 2 shows the basic geometry of the photobioreactor cell according to an embodiment of the invention in a schematic side view of the modular photobioreactor cell. The height dimensions of individual photobioreactor cells may be varied to optimise growth conditions within the cell, or the height may be varied to optimise power transfer conditions for device performance, or the height may be varied to optimise light transfer conditions for device performance, or the height may be varied to optimise hydrodynamics and/or gas dynamics. The height of the photobioreactor cell may be between, but not limited to, 1,000 mm and 10,000 mm. Individual photobioreactor cells may vertically stacked and directly interconnected to other photobioreactor cells using the ports, and/or may be indirectly interconnected using a connection module. The connection module may enable additional power and/or light to be delivered to the photobioreactor cells, and/or, may enable additional gas and/or liquids to be delivered into the liquid medium within the photobioreactor cell.

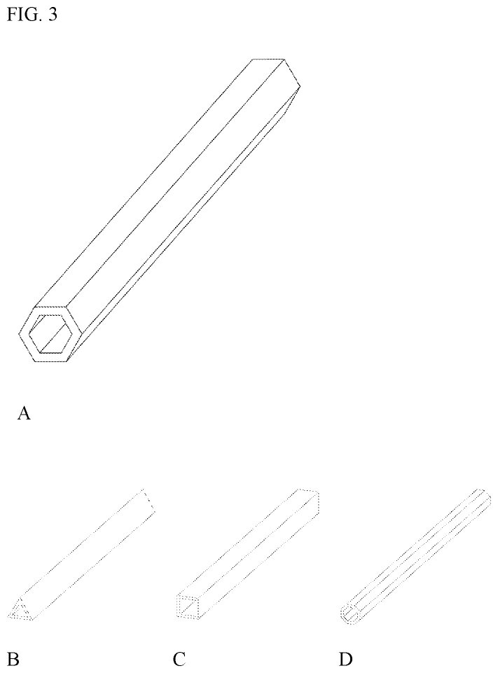

FIG. 3 shows the basic geometry of the photobioreactor cell according to an embodiment of the invention in a schematic isometric perspective view of the modular photobioreactor cell. The photobioreactor cells are modular and can be assembled into larger systems comprised of passive and/or active devices. The cells can be connected horizontally and vertically to form optimised systems. The luminous modular cells can be of different functions including a passive light guide or wave guide cell, a passive low power light guide or wave guide cell, an active light-emitting diode (LED) cell, or a wide range of active emissive technologies including organic light-emitting diode (OLED) or polymer light-emitting diode (PLED, P-OLED) or active-matrix organic light emitting diode (AM-OLED) or electro-emissive quantum dot light emitting diode (QD-LED, ELQD, EL-QLED, QDEL) or photo-emissive quantum dot light emitting diode (QLED, QD-OLED).

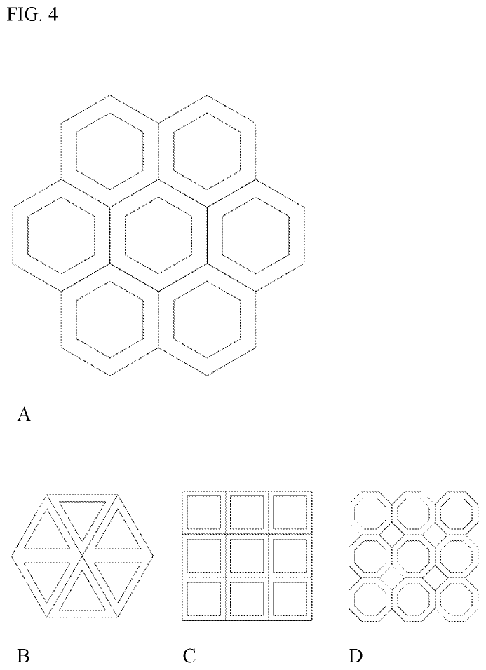

FIGS. 4A-E show top view of several array configurations according to embodiments of the invention of multiple photobioreactor cells in a schematic top/bottom view of different photobioreactor cell geometries with hexagonal polygon, equilateral triangular, quadrilateral polygon and octagonal polygon cross-sectional shape.

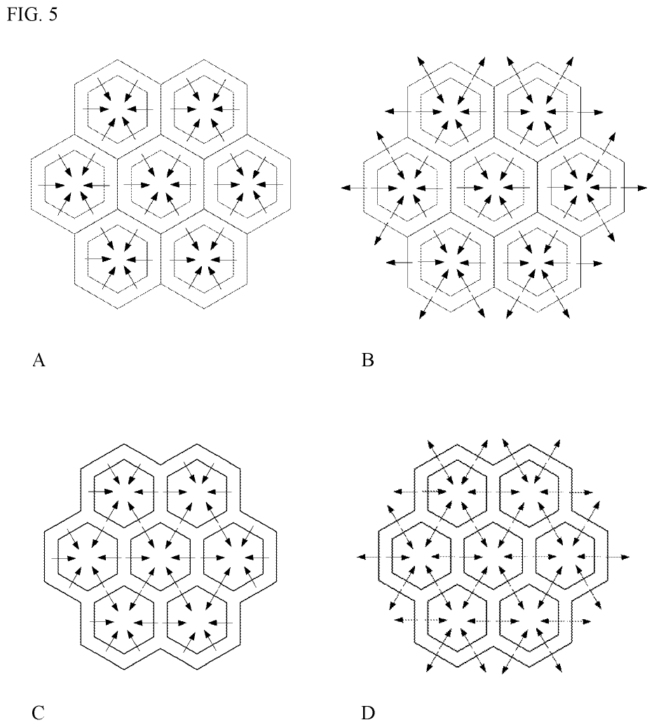

FIGS. 5A-D show schematic top/bottom views of several array configurations of multiple photobioreactor hexagonal cells according to embodiments of the invention with different configurations of cell walls, and with different configurations of light emission directionality. FIGS. 5A-B show embodiments of a photobioreactor cell array assembly comprised of seven hexagonal cells with a two cell wall internal array structure. The two cell wall internal array structure may have advantages due to the reduced complexity of manufacturing and assembly of arrays, that may be comprised of active and/or passive devices, and the two cell wall internal array structure may have advantages for operation and maintenance of the individual photobioreactor cells or the arrays. The two cell wall internal array structure may have disadvantages due to increased costs of materials for manufacturing and fabrication of array structures. FIGS. 5C-D show embodiments of a photobioreactor cell array assembly comprised of seven hexagonal cells with a one cell wall internal array structure. The one cell wall internal array structure may have disadvantages due to the increased complexity of manufacturing and assembly of the arrays, that may be comprised of active and/or passive devices, and the one cell wall internal array structure may have disadvantages for operation and maintenance of the individual photobioreactor cells or the array. The one cell wall internal array structure may have advantages for decreased costs of materials for manufacturing of array structures. FIGS. 5A and 5C show embodiments of the photobioreactor arrays comprised of photobioreactor cells that may emit light in a single direction through the internal surface or the external surfaces of the cells. FIGS. 5B and 5D show embodiments of the photobioreactor arrays comprised on photobioreactor cells that may emit light bidirectionally through the internal and external surfaces of the cells. Single directional light emission through the internal surface of photobioreactor cell may have advantages of lower power requirements and/or lower light intensity requirements compared to bidirectional illumination. Single directional light emission may have disadvantages of lower power efficiency and/or lower light emission efficiency due to light attenuation by layers within the thin film device. Bidirectional light emission through the internal surface and the external surface of photobioreactor cell may have disadvantages of higher power requirements and/or higher light intensity requirements compared to single directional illumination. Bidirectional light emission may have advantages of higher power efficiency and/or higher light emission efficiency due to decreased light attenuation by layers within the thin film device.

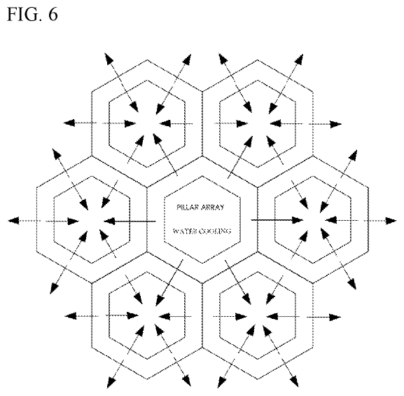

FIG. 6 shows a top/bottom view of an embodiment of a pillar array configuration according to an embodiment of the invention of multiple photobioreactor cells illustrating one possible pillar array configuration of a central light emitting cell surrounded by light guide cells or wave guide cells. The cells may be actively coupled and interconnected using ports embedded within the surface of the cell walls. The cells can be passively coupled and interconnected through cell wall surface proximity without using ports. The cells may have different optical characteristics including refractive indices, reflectiveness, transparency (translucency and opacity) to allow for the control, switching, transmission, emission and scattering of light. The cell walls may be conductive to allow for the transmission of power. The LED pillar is configured to emit light through the external surface into the surrounding cells. The internal core of the LED pillar is configured for water cooling to manage waste heat from semiconductor based illumination technology and regulate temperature.

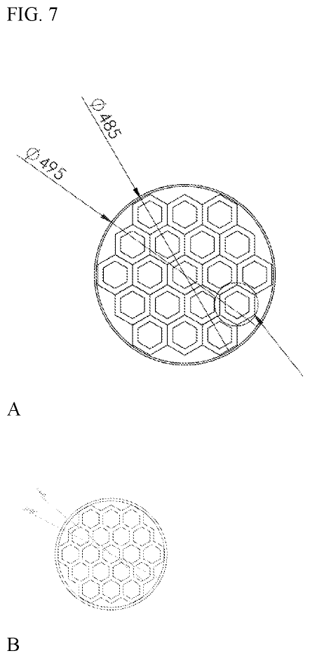

FIG. 7 shows a top/bottom view of embodiments of an array module configuration according to an embodiment of the invention of multiple hexagonal photobioreactor cells within a protective structure illustrating possible array module configurations. The protective structure may be made of different materials including metals, ceramics, polymers, others that protect the cells from operational stresses and from maintenance stresses. The number of photobioreactor cells in the array module can be scaled horizontally and/or vertically.

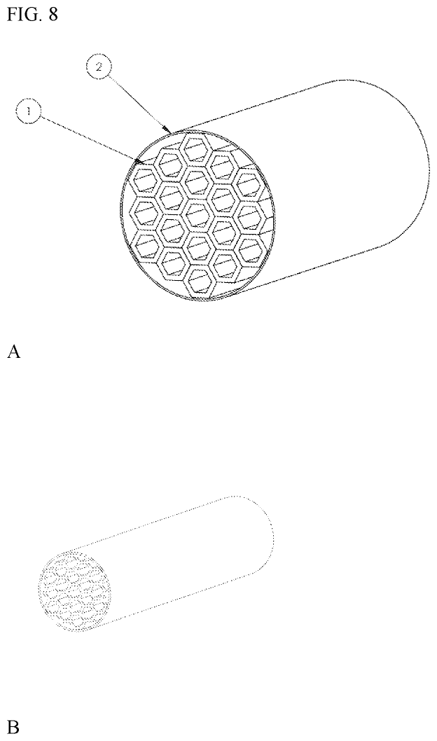

FIG. 8 shows an isometric view of embodiments of an array module configuration according to an embodiment of the invention of multiple photobioreactor cells within a protective structure is of multiple photobioreactor cells contained within a protective structure illustrating possible array module configurations. The array module may be configured in a similar way as tubular membranes for horizontal or vertical flows. The modules can be deployed and operated in different configurations including horizontal operation, vertical operation, and coiled operation. Horizontal operation may be beneficial for liquid flow. Vertical operation may be beneficial for gas flow. Coiled configuration may be beneficial for both liquid flow and gas flow.

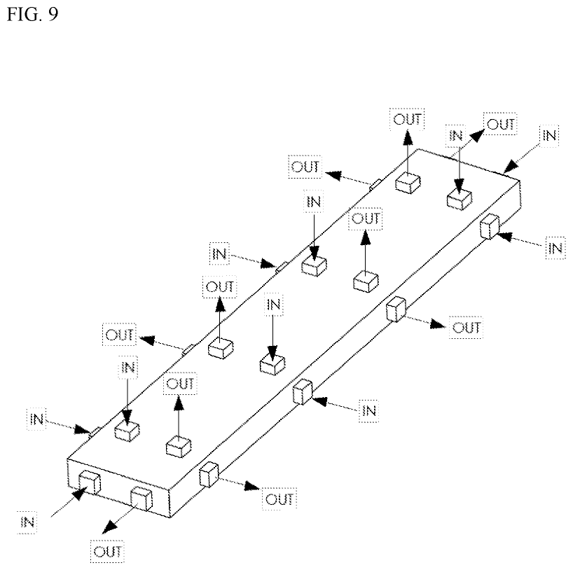

FIG. 9 shows an isometric view of an embodiment of a cell wall of a light guide showing the optical input ports and optical output ports within the cell wall for the transmission of light, in order for light to travel between connected panels, and diffuse into the medium. The optical interconnect ports (e.g., input ports) may be connected to fibre optic cables to provide input light. The optical interconnect ports can be used to provide light from one cell to another connected cell.

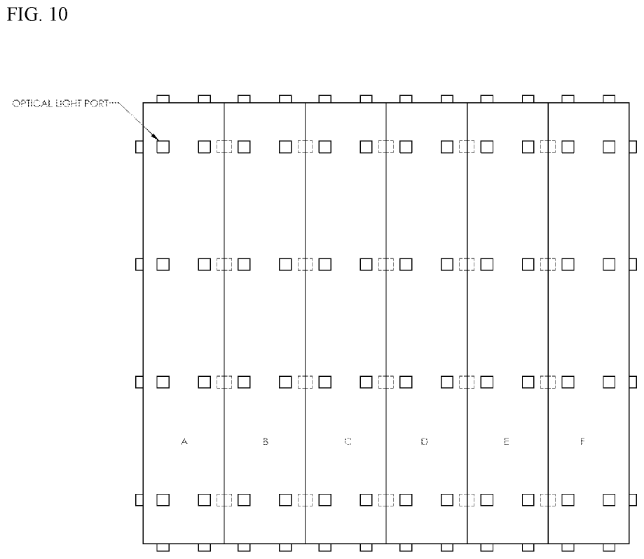

FIG. 10 shows an embodiment of the invention having connected cell walls of a thin film hexagonal light guide cell, prior to being fully connected into a polygonal prism. The cell walls may be interconnected along the long side to each other, such that light entering from the input port at the top travels within the cell and is transmitted between the cell walls, or transmitted between photobioreactor cells, as well as diffused along the planar surface into the medium.

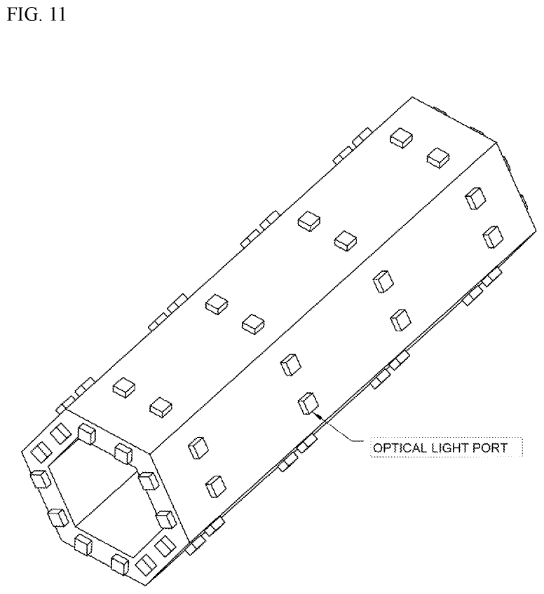

FIG. 11 shows an isometric view of an embodiment of a thin film hexagonal light guide cell in a polygonal prism shape. The light guide cells can be interconnected to other cells, allowing light to be transferred between the photobioreactor cells. The internal and/or external surfaces of the light guide cell may emit light into the medium. The entire external surface of the cell can emit light. The internal surface of the cell can be patterned to enable different light wavelengths to be emitted across the surface pattern.

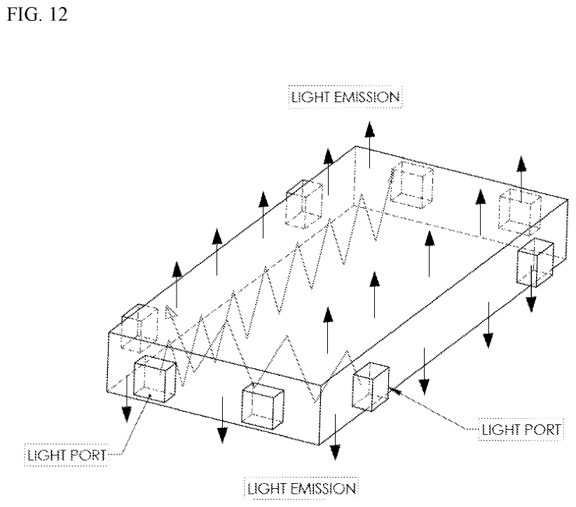

FIG. 12 shows an isometric view of an embodiment of a thin film light guide cell wall illustrating schematic ray tracing of light transmission and emission from within the light guide. The thin film light guide may be a solid state device, which may be programmable to allow transmission and emission of light to be digitally controlled. Digital control of the light guide device may allow light emission intensity to be controlled within the light guide cell, and may allow light transmission to be controlled and directed between light guide cells. According to embodiments of the invention, digital control of light may include, for example, pulse width modulation or other signal control techniques.

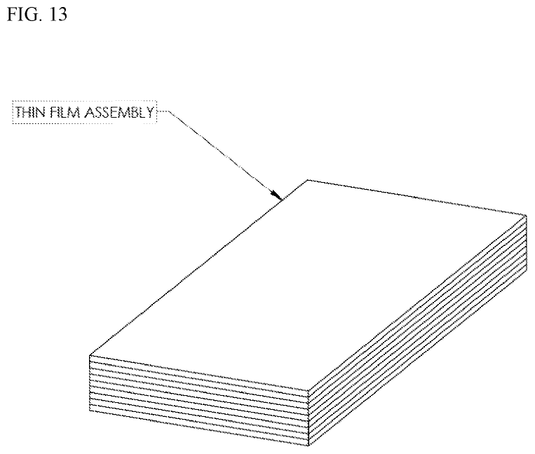

FIG. 13 shows a isometric view of an embodiment of a thin film assembly of structural and functional layers of a light guide device. The device may be encapsulated using an encapsulant material to protect the device from the environment. The encapsulant may provide resistance and protection from a range of environmental conditions and stresses including gases, aqueous liquids, organic liquids, temperature, and pH. The encapsulant may be rigid or flexible.

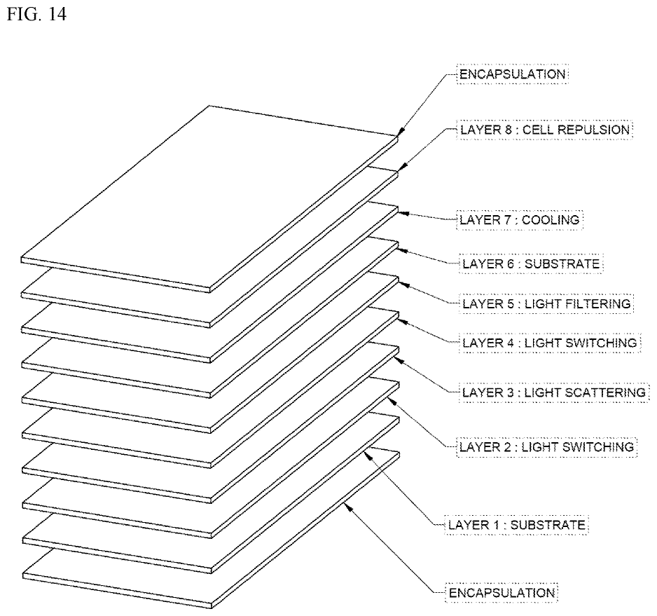

FIG. 14 shows an isometric view of an embodiment of a thin film light guide illustrating individual functional and structural layers of the light guide. It will be understood that this is an exemplary list of layers, and not all layers must be present in every embodiment of the invention.

FIG. 14 LAYER 1: shows a substrate layer that may provide the base material for deposition of the other functional layers to the device. The substrate may be rigid or flexible.

FIG. 14 LAYER 2: shows a light switching layer that may control the transmission and direction of light. The light switching layers may direct light into the light scattering layer for light emission. The light switching layer may direct the light out of the device through the optical ports. The light switching layer may be patterned for optical circuits. The light switching layer may use materials such as liquid crystals, nanomaterials or mirrors.

FIG. 14 LAYER 3: shows a light scattering layer that may control the emission of light in many directions. The light scattering layer can use materials such as nanocrystals and/or nanocomposites.

FIG. 14 LAYER 4: shows a light switching layer that may control the transmission and direction of light. The light switching layers may direct light into the light scattering layer for light emission. The light switching layer may direct the light out of the device through the optical ports. The light switching layer may be patterned for optical circuits. The light switching layer may use materials such as liquid crystals, nanomaterials or mirrors.

FIG. 14 LAYER 5: shows a light filtering layer that may control the wavelengths of light. The light filtering layer may filter specific wavelengths of light that are unable to be utilised for photosynthesis. The light filtering layer may up shift the light wavelengths, and/or may down shift the light wavelengths. The light wavelengths may be optimised for the photosynthetic active region of visible light. The light wavelengths may be finely tuned for different photosynthetic pigments, and/or may be optimised for light absorption by the photosynthetic microorganisms.

FIG. 14 LAYER 6: shows a substrate layer that may provide the base material for deposition of the other functional layers to the device. The substrate may be rigid or flexible.

FIG. 14 LAYER 7: shows a cooling layer that may maintain stable operating temperatures of the device to optimised device performance. The cooling layer may be used to lower the temperature of the device which interfaces with the liquid. The lowered surface temperature may be used to reduce biological adherence (fouling). The cooling layer may use materials such as functionalised graphene or carbon nanotubes.

FIG. 14 LAYER 8: shows a cell adhesion repulsion layer that may attract or repulse microorganisms from the surface of the device. The adherence of some photosynthetic microorganisms to the light emitting surface may optimise growth conditions and productivity, the adherence of some photosynthetic microorganisms may optimise recovery of the biomass for downstream processing. The repulsion of some photosynthetic microorganisms from the light emitting surface may optimise growth conditions and productivity, the repulsion of some microorganisms may optimise recovery of the biomass for downstream processing. The cell adhesion repulsion layer may use materials such as conductive metals and polymers.

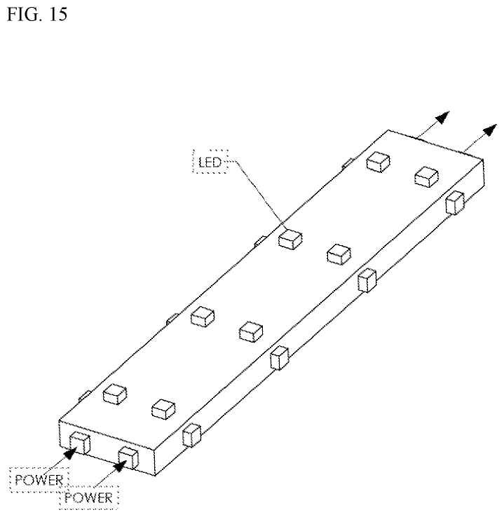

FIG. 15 shows an isometric view of an embodiment of a thin film LED cell wall with power interconnect ports and the LED packages within the cell wall for light emission. The power interconnect ports can be used to connect one cell to another cell. The LED packages can be used to interconnect to light guide cells allowing light to be transferred from an active light emission device to a passive light guide device.

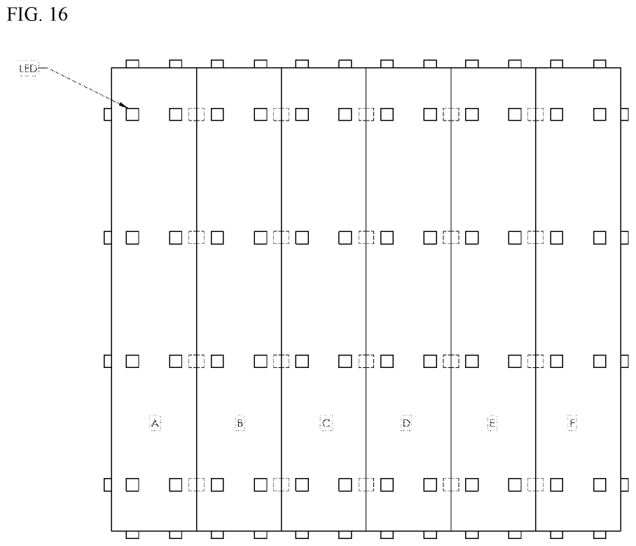

FIG. 16 shows an isometric view of an embodiment of cell walls of a thin film hexagonal LED cell. The cell walls may be optically interconnected connected to each other within the cell allowing light to be transmitted between the cell walls, or transmitted between photobioreactor cells.

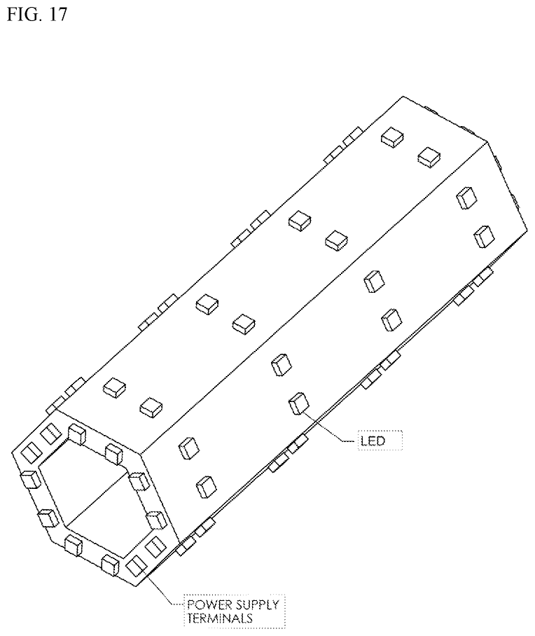

FIG. 17 shows an isometric view of a thin film hexagonal LED cell according to embodiment of the invention. The LED cells can be interconnected to other photobioreactor cells, allowing light to be transferred between the photobioreactor cells. In some embodiments of the invention, the external surfaces of the LED cell may emit light. In some embodiments of the invention, the entire external surface of the cell may emit light.

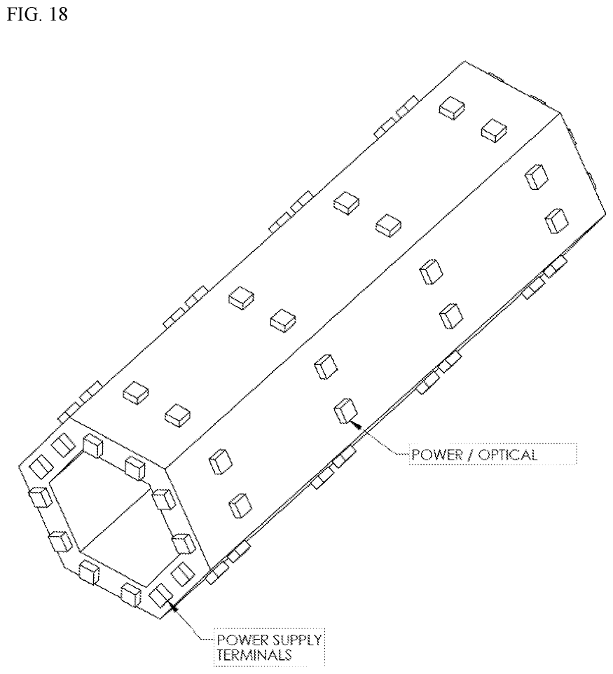

FIG. 18 shows a isometric view of an embodiment of a thin film hexagonal OLED, PLED or QD-LED cell. The active device may be fabricating using a range printable emissive technologies including AM-OLED, QD-LED, ELQD, EL-QLED, QDEL, QLED, QD-OLED, nanoLED or microLED. In some embodiments of the invention, the entire internal and/or external surfaces of the photobioreactor cell may emit light. The embodiment may have optical light ports within the cell wall for the transmission of light. In some embodiments of the invention, the optical interconnect ports may be connected to fibre optic cables. In some embodiments of the invention, the optical interconnect ports may be used for active coupling to transfer light from one photobioreactor cell to another photobioreactor cell. In some embodiments of the invention, the surface of the photobioreactor cell wall may be used for active coupling to transfer light from one photobioreactor cell to another photobioreactor cell. In some embodiments of the invention, the power interconnect ports may be used for active coupling to transfer power from one photobioreactor cell to another photobioreactor cell.

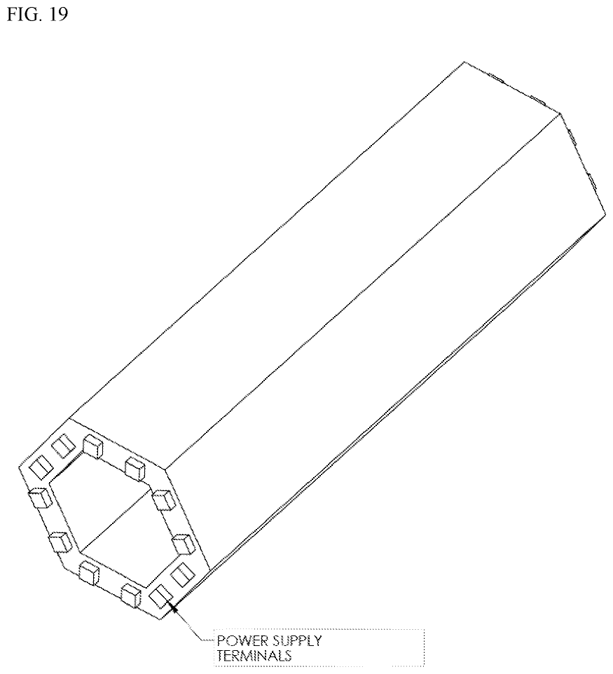

FIG. 19 shows a isometric view of a thin film hexagonal OLED, PLED or QD-LED cell according to embodiments of the invention. The active device may be fabricating using a range printable emissive technologies including AM-OLED, QD-LED, ELQD, EL-QLED, QDEL, QLED, QD-OLED, nanoLED, or microLED. In some embodiments of the invention, the entire internal and/or external surfaces of the photobioreactor cell may emit light. The OLED, PLED or QD-LED device may be passively coupled to transfer light from an active emitter photobioreactor cell to a passive light guide photobioreactor cell. The power interconnect ports may be used for active coupling to transfer power from one photobioreactor cell to another photobioreactor cell.

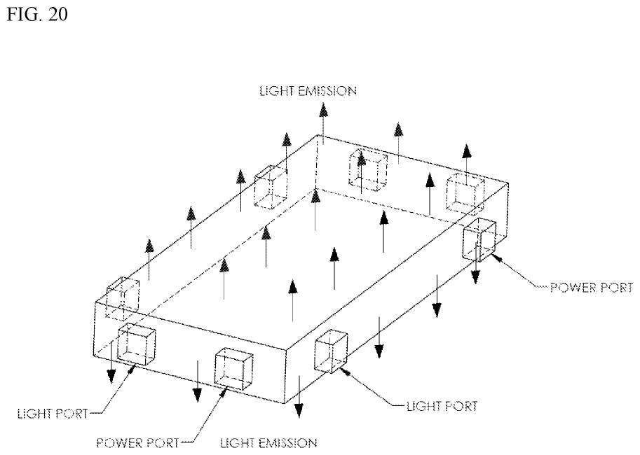

FIG. 20 shows an isometric view of a thin film OLED, PLED or QD-LED cell wall according to embodiments of the invention, illustrating schematic ray tracing of light transmission and emission from within the light guide. The thin film cell may be a solid state device. The device may be programmable allowing transmission and emission to be digitally controlled. Digital control of the light guide device may allow light emission intensity to be controlled within the light guide cell, and may allow light transmission to be controlled and directed between light guide cells. According to embodiments of the invention, digital control of light may include, for example, pulse width modulation or other signal control techniques.

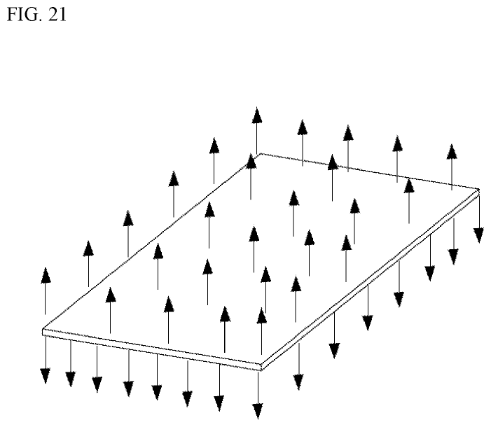

FIG. 21 shows an isometric view of an embodiment of a thin film OLED, PLED or QD-LED cell wall illustrating bidirectional emission of light from both surfaces of the thin film.

Shows a isometric view of an embodiment of a thin film assembly of structural and functional layers of a OLED, PLED or QD-LED device. The device may be encapsulated using an encapsulant material to protect the device from the environment. The encapsulant may provide resistance and protection from a range of environmental conditions and stresses including gases, aqueous liquids, organic liquids, temperature, and pH. The encapsulant may be rigid or flexible. It will be understood that this is an exemplary list of layers, and not all layers must be present in every embodiment of the invention.

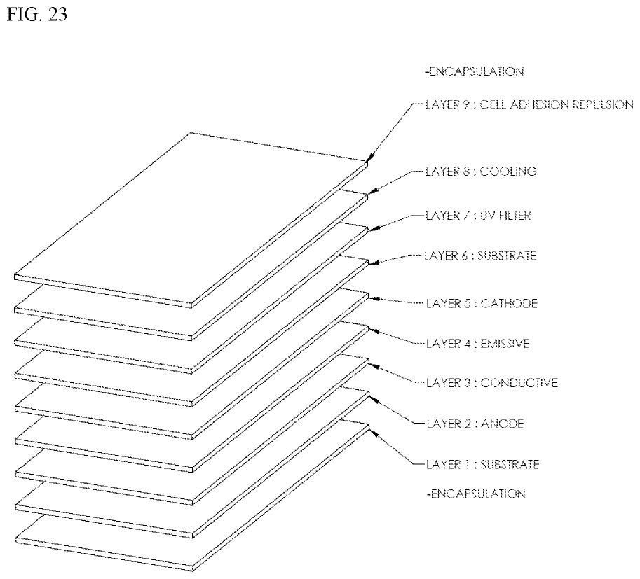

FIG. 23 LAYER 1: shows a substrate layer that may provide the base material for deposition of the other functional layers to the device. The substrate may be rigid or flexible.

FIG. 23 LAYER 2: shows an anode layer that may be positively charged to the organic or polymer layers and functions to remove electrons from the device when current is applied. The anode layer may use materials such as indium tin oxide (ITO).

FIG. 23 LAYER 3: shows a conductive layer that may control the transport of holes from the anode layer. The conductive layer may use materials such as polymers, polyaniline, polyethylenedioxythiophene (PEDOT), and/organic molecules like graphene and carbon nanotubes.

FIG. 23 LAYER 4: shows an emissive layer that may control the emission of light. The emissive layer may use one or more organic molecules, polymers or nanocrystals for the emission of light. The emissive layer may use materials such as organic molecules, polymers or nanoscrystals. There are a wide range of emissive molecules and structures available.

FIG. 23 LAYER 5: shows a cathode layer that may control the injection of electrons into the device. The conductive layer may use materials such as conducting oxides, carbon nanotubes or silver nanowires.

FIG. 23 LAYER 6: shows a substrate layer that may provide the base material for deposition of the other functional layers to the device. The substrate may be rigid or flexible.

FIG. 23 LAYER 7: shows an ultraviolet (UV) light filtering layer that may block UV light. The UV light filters may protect the materials within the device. UV light may be used for cleaning of the device surface to remove fouling. The light filtering layer may use materials such as acrylics.

FIG. 23 LAYER 8: shows a light filtering layer that may control the wavelengths of light. The light filtering layer may filter specific wavelengths of light that are unable to be utilised for photosynthesis. The light filtering layer may up shift the light wavelengths, and/or may down shift the light wavelengths. The light wavelengths may be optimised for the photosynthetic active region of visible light. The light wavelengths may be finely tuned for different photosynthetic pigments, and/or may be optimised for light absorption by the photosynthetic microorganisms.

FIG. 23 LAYER 9: shows a cell adhesion repulsion layer that may attract or repulse microorganisms from the surface of the device. The adherence of some photosynthetic microorganisms to the light emitting surface may optimise growth conditions and productivity, the adherence of some photosynthetic microorganisms may optimise recovery of the biomass for downstream processing. The repulsion of some photosynthetic microorganisms from the light emitting surface may optimise growth conditions and productivity, the repulsion of some microorganisms may optimise recovery of the biomass for downstream processing. The cell adhesion repulsion layer may use materials such as conductive metals and polymers.

The OLED, PLED or QD-LED cell device functionality may be increased by the inclusion of additional structural and/or functional layers.

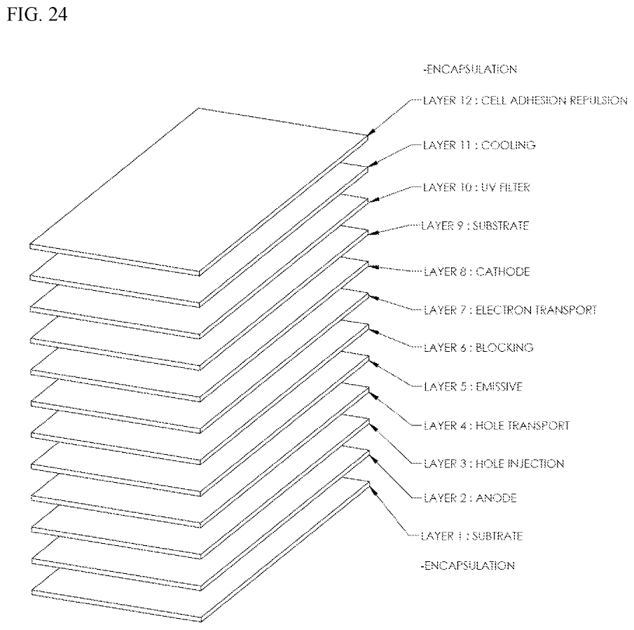

FIG. 24 Shows an isometric view of an embodiment of a thin film OLED, PLED or QD-LED device illustrating individual functional and structural layers of the light guide. It will be understood that this is an exemplary list of layers, and not all layers must be present in every embodiment of the invention.

FIG. 24 LAYER 1: Shows a substrate layer that may provide a base material for deposition of the other functional layers to the device. The substrate may be rigid or flexible.

FIG. 24 LAYER 2: Shows a anode layer that may be positively charged to the organic or polymer layers, and functions to remove electrons from the device when current is applied. The anode layer may use conductive materials such as indium tin oxide (ITO).

FIG. 24 LAYER 3: Shows a hole injection layer that may control the extraction of electron holes from the anode and inject the electron holes into the hole transport layer. The hole injection layer may provide structural and functional optimisations for improved device performance. The hole injection layer may use materials such as poly(3,4-ethylenedioxythiophene):poly(styrene sulfonate) (PEDOT:PSS).

FIG. 24 LAYER 4: Shows the hole transport layer that may control the transport of electron holes from the hole injection layer to the emissive layer. The hole transport layer provides structural and functional optimisations for improved device performance. The blocking layer may use materials such as organic molecules or polymers.

FIG. 24 LAYER 5: Shows an emissive layer that may control the emission of light. The emissive layer may use one or more organic molecules, polymers or nanocrystals for the emission of light. The emissive layer may use materials such as organic molecules, polymers or nanocrystals. There are a wide range of emissive molecules and structures available.

FIG. 24 LAYER 6: Shows a blocking layer that may control the charge carriers to the emissive layer. The hole transport layer provides structural and functional optimisations for improved device performance. The blocking layer may use materials such as organic molecules and/or polymers.

FIG. 24 LAYER 7: shows the electron transport layer the controls the transport of electrons from the cathode to blocking layer. The electron transports layer can use a wide range of materials and/or dopants.

FIG. 24 LAYER 8: Shows a cathode layer that may control the injection of electrons into the device. The conductive layer may use materials such as conducting oxides, carbon nanotubes or silver nanowires.

FIG. 24 LAYER 9: Shows a substrate layer that may provide a base material for deposition of the other functional layers to the device. The substrate may be rigid or flexible.

FIG. 24 LAYER 10: Shows an ultraviolet (UV) light filtering layer that may block UV light. The UV light filters may protect the materials within the device. UV light may be used for cleaning of the device surface to remove fouling. The light filtering layer may use materials such as acrylics.

FIG. 24 LAYER 11: Shows a cooling layer that may maintain stable operating temperatures of the device to optimised device performance. The cooling layer may be used to lower the temperature of the device which interfaces with the liquid. The lowered surface temperature may be used to reduce biological adherence (fouling). The cooling layer may use materials such as functionalised graphene or carbon nanotubes.

FIG. 24 LAYER 12: Shows a cell adhesion repulsion layer that may attract or repulse microorganisms from the surface of the device. The adherence of some photosynthetic microorganisms to the light emitting surface may optimise growth conditions and productivity, the adherence of some photosynthetic microorganisms may optimise recovery of the biomass for downstream processing. The repulsion of some photosynthetic microorganisms from the light emitting surface may optimise growth conditions and productivity, the repulsion of some microorganisms may optimise recovery of the biomass for downstream processing. The cell adhesion repulsion layer may use materials such as conductive metals and polymers.

The OLED, PLED, QD-LED cell device functionality may be increased by the inclusion of additional structural and/or functional layers.

1. An illumination system for use in a photobioreactor containing photosynthetic microorganisms, comprising
an array of polygonal prism cells, each of said polygonal prism cells comprising a plurality of substantially flat rectangular panels, each panel having a top edge, a bottom edge, and lateral edges and a flat surface therebetween,
wherein said panels are configured to provide illumination at said flat surface, and further configured to be connected to at least one other of said panels along each said lateral edge, and
wherein the top and bottom of the cells formed by the respective top and bottom edges of the panels thereof are substantially open, thereby permitting flow therethrough of a fluid containing said photosynthetic microorganisms to be illuminated at the flat surfaces of said panels.

2. The illumination system of claim 1, wherein the flat surfaces of said panels are made of a plurality of interior layers and an exterior layer comprising a transparent material permitting light to pass therethrough, said transparent material selected from the group consisting of glass, ceramic, a polymer, an acrylic, a composite material, and any combination thereof.

3. The illumination system of claim 2, wherein at least one of said interior layers is a wavelength filtering layer and/or wavelength shifting layer.

4. The illumination system according to any of the foregoing claims, wherein at least one of said panels is a passive panel, said passive panel being configured to receive light energy from a light source external to said panel and to diffuse said light energy through the flat surface thereof.

5. The illumination system of claim 4, wherein at least one of said interior layers is a light scattering layer for diffusing light.

6. The illumination system according to any of claims 1-3, wherein at least one of said panels is an active panel, comprising an light emitting layer, said light emitting layer including one or more light emitting elements.

7. The illumination system of claim 6, wherein the light emitting element comprises at least one element selected from the group consisting of: a light emitting diode (LED), a printable organic light emitting diode (OLED), a printable polymer light emitting diode (PLED), and a printable quantum dot light emitting diode (QD-LED).

8. The illumination system according to any of the foregoing claims, wherein the array includes polygonal prism cells having cross-sectional shapes selected from the group consisting of triangular, rectangular, hexagonal, and octagonal, and any combination thereof.

9. The illumination system according to any of the foregoing claims, wherein the array of polygonal prism cells is formed by a plurality of such cells arranged longitudinally alongside each other by connecting lateral sides thereof, wherein the array of cells is surrounded by a protective structure.

10. The illumination system of any of the foregoing claims, further comprising an auxiliary system in fluid communication with the array of polyhedron prism cells for causing the fluid containing said photosynthetic microorganism to flow through the array of tubular polyhedron prism cells.

11. The illumination system of any of the foregoing claims, wherein the flat surface of at least one of said panels is configured to provide illumination both sides of said flat surface.

12. The illumination system of any of the foregoing claims, wherein at least one of said panels is configured to receive light energy at a light input port at a first edge thereof, and to transfer said light energy from said light input port to a flat panel thereof and/or to a light output port at a second edge of said panel.

13. The illumination system of any of the foregoing claims, wherein at least one of said panels is configured to receive light energy at a light input port at one side of a flat surface thereof, and to transfer at least some of said light energy from said light input port to the other side of said flat surface of said panel and/or to a light output port at an edge of said panel.
